(19) United States
(12) Patent Application Publication
(10) Pub. No.: US 2017/0035626 A1
(43) Pub. Date: Feb. 9, 2017

(54) CAST COVER

(71) Applicant: Eduardo Rubino, Basking Ridge, NJ (US)

(72) Inventor: Eduardo Rubino, Basking Ridge, NJ (US)

(21) Appl. No.: 14/817,404

(22) Filed: Aug. 4, 2015

(51) Int. Cl. A61F 15/00; A61F 13/04

(52) U.S. Cl. CPC A61F 15/004; A61F 13/043; A61F 13/04

(57) ABSTRACT

A cover that is sized to secure over an appendage of an animal, such as a human is provided. The cover includes a tubular sidewall. The sidewall has a closed bottom end and an open top end formed by an upper rim. The sidewall further includes a first inner edge and a second inner edge formed along a length of the tubular sidewall and terminating at the upper rim. A first connector releasably connects the first inner edge and the second inner together, forming a water tight seal along the length of the tubular sidewall.

### BACKGROUND OF THE INVENTION

The present invention relates to protecting casts and, more particularly, to a cover for a cast.

An orthopedic cast, body cast, plaster cast, or surgical cast, is a shell, frequently made from plaster, encasing a limb (or, in some cases, large portions of the body) to stabilize and hold anatomical structures, most often a broken bone (or bones), in place until healing is confirmed. It is similar in function to a splint.

To prevent mold from growing within the cast, a cover is typically used to protect the cast while taking a bath or shower. Existing covers require stretching of a rubber seal in order to hold the cover open and to insert the cast. This requires the assistance of another person and often bends the injured limb risking further injury.

As can be seen, there is a need for an improved cover that may used without the assistance of another individual.

### SUMMARY OF THE INVENTION

In one aspect of the present invention, a cover sized to secure over an appendage of an animal comprises: a tubular sidewall comprising a closed bottom end, an open top end formed by an upper rim, and a first inner edge and a second inner edge formed along a length of the tubular sidewall and terminating at the upper rim; a first connector releasably connecting the first inner edge and the second inner together, forming a water tight seal along the length of the tubular sidewall; an elastic flexible band attached to the upper rim and comprising a first end disposed near the first inner edge, and a second end disposed near the second inner edge; and a second connector releasably connecting the first end and the second end of the elastic flexible band together.

In another aspect of the present invention, a method of covering a cast of an injured animal comprises: providing a cover comprising: a tubular sidewall comprising a closed bottom end, an open top end formed by an upper rim, and a first inner edge and a second inner edge formed along a length of the tubular sidewall and terminating at the upper rim; and an elastic flexible band attached to the upper rim and comprising a first end disposed near the first inner edge, and a second end disposed near the second inner edge; placing the cover over the cast; releasably connecting the first inner edge and the second inner edge together by a first connector, forming a water tight seal along the length of the tubular sidewall; releasably connecting the first end and the second end of the elastic flexible band together by a second connector

These and other features, aspects and advantages of the present invention will become better understood with reference to the following drawings, description and claims.

### BRIEF DESCRIPTION OF THE DRAWINGS

FIG. 1 is a perspective view of an embodiment of the present invention shown after application and fully secured;

FIG. 2 is an exploded view of an embodiment of the present invention shown pre-application;

FIG. 3 is a perspective view of an embodiment of the present invention shown post application and unsecured;

FIG. 4 is a perspective view of the present invention shown post application and partially secured;

FIG. 5 is a section view of an embodiment of the present invention along line 5-5 in FIG. 1; and

FIG. 6 is a section detail view of an embodiment of the present invention along line 6-6 in FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a zippered cover to protect a cast or bandage while taking a shower. The present invention allows a person with an injured arm or leg to cover their cast or bandage for the purpose of taking a shower or bath without assistance from another person or risking further injury.

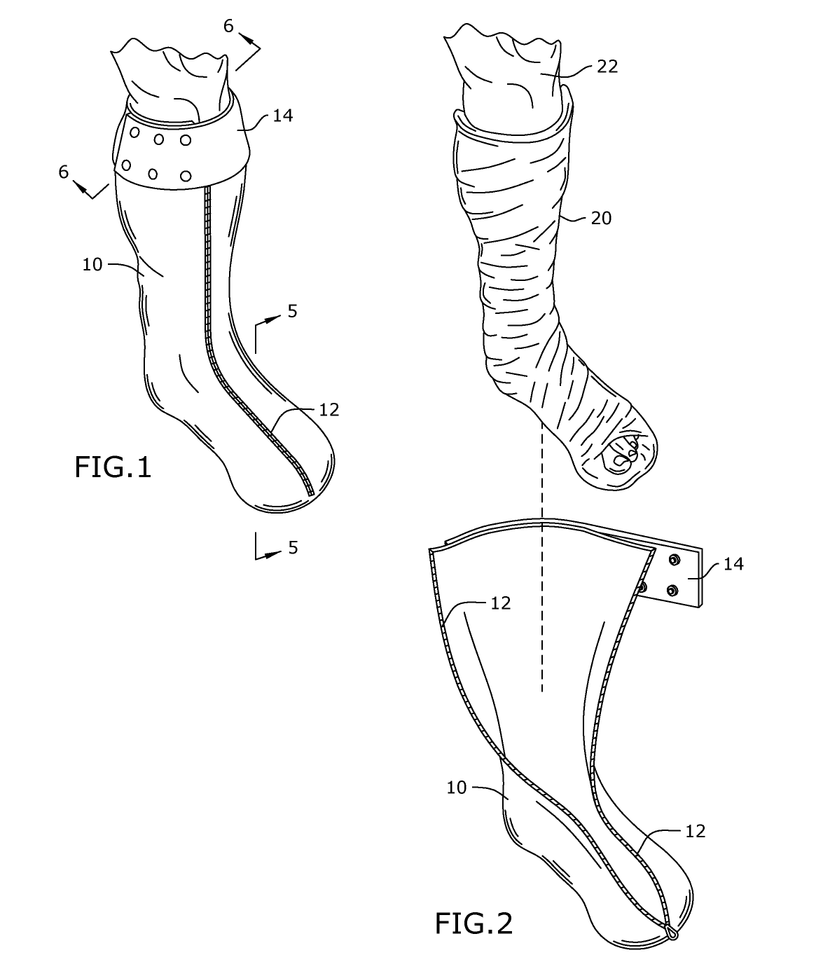

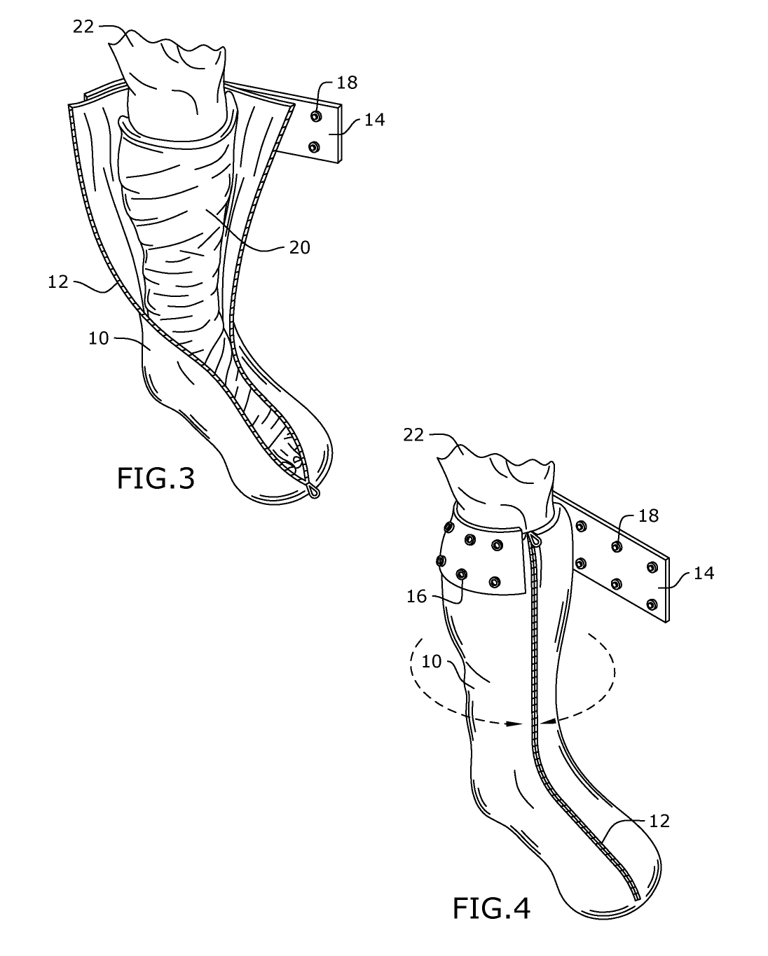

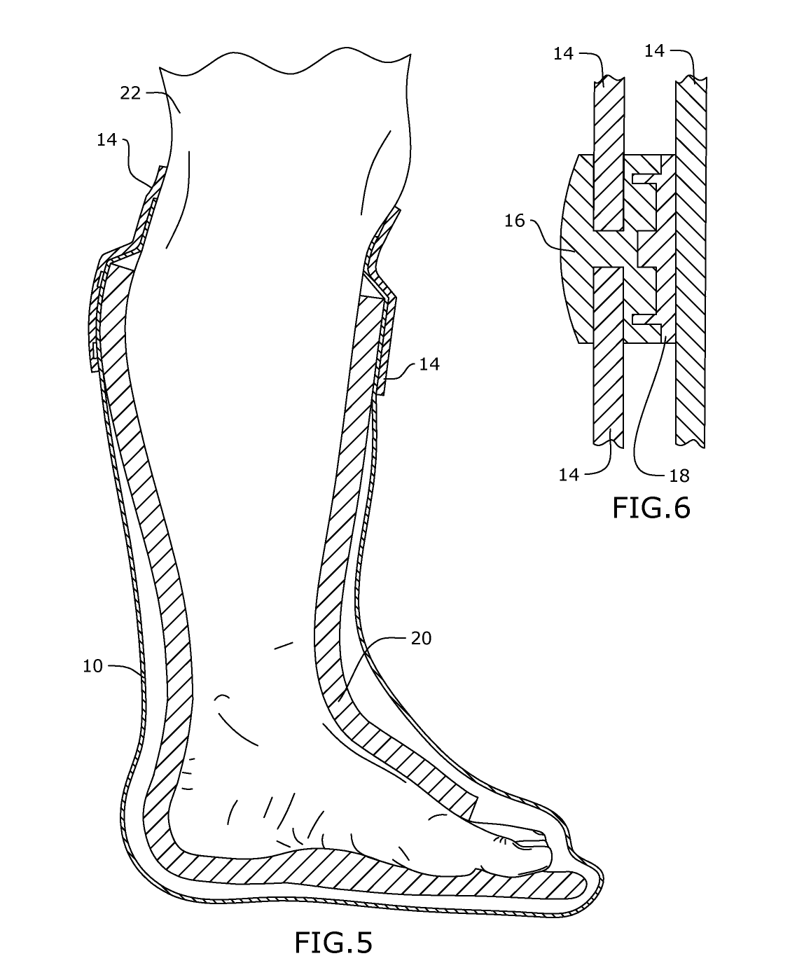

Referring to FIGS. 1 through 6, the present invention includes a cover that is sized to secure over an appendage of an animal, such as a human 22. The cover includes a tubular sidewall 10. The sidewall 10 has a closed bottom end and an open top end formed by an upper rim. The sidewall 10 further includes a first inner edge and a second inner edge formed along a length of the tubular sidewall 10 and terminating at the upper rim. A first connector releasably connects the first inner edge and the second inner together, forming a water tight seal along the length of the tubular sidewall 10.

In certain embodiments, the present invention may include an elastic flexible band 14. The band 14 is fixed to the upper rim of the tubular sidewall 10. In certain embodiments, the band 14 is attached to a substantial portion of the upper rim. The band 14 includes a first end and a second end. The first end is disposed near the first inner edge, and a second end disposed near the second inner edge. A second connector releasably connects the first end and the second end of the elastic flexible band 14 together.

The first inner edge and the second inner edge may run from the bottom closed end up to the open top end. The first inner edge and the second inner edge may face one another and may be connected together by the first connecter. The first connector may be a zipper 12. The zipper 12 may form a water tight seal, preventing water from leaking into the cover and onto the cast 20.

The second connector may include a plurality of male snap buttons 18 and a plurality of female snap buttons 16. The second connector may be different types of connectors, such as clips, hook and loop fasteners, and the like. The plurality of male snap buttons 18 may be disposed on an inner surface of the first end of the band 14. The plurality of female snap buttons 16 may be disposed on an outer surface of the second end of the band 14. In certain embodiments, one of the female and male snap buttons 16, 18 may be arranged in rows about the circumference of band 14, allowing the band size to be adjustable.

As illustrated in the Figures, the sidewall 14 may be shaped to fit over a foot. However, the sidewall 14 may be shaped to fit over arms, legs, hands, and fingers. The sidewall 14 is formed of a water impermeable material. The elastic flexible band is made of a polymer. The polymer may be rubber. Since the band 14 is flexible and elastic, the band 14 may be wrapped around the appendage to form a water tight seal, preventing water from entering through the open top end.

A method of covering a cast 20 of an injured animal may include the following. The cover mentioned above may be placed over the cast 20. The first inner edge and the second inner edge are releasably connected together by a first connector, such as a zipper 12, forming a water tight seal along the length of the tubular sidewall 10. The first end and the second end of the elastic flexible band may be releasably connected together by a second connector, such as snap buttons 16, 18, forming a water tight seal along the upper rim of the cover.

It should be understood, of course, that the foregoing relates to exemplary embodiments of the invention and that modifications may be made without departing from the spirit and scope of the invention as set forth in the following claims.

What is claimed is:

1. A cover sized to secure over an appendage of an animal comprising:
a tubular sidewall comprising a closed bottom end, an open top end formed by an upper rim, and a first inner edge and a second inner edge formed along a length of the tubular sidewall and terminating at the upper rim;
a first connector releasably connecting the first inner edge and the second inner together, forming a water tight seal along the length of the tubular sidewall;
an elastic flexible band attached to the upper rim and comprising a first end disposed near the first inner edge, and a second end disposed near the second inner edge; and
a second connector releasably connecting the first end and the second end of the elastic flexible band together.

2. The cover of claim 1, wherein the first connector is a zipper running from the closed bottom end to the open top end.

3. The cover of claim 1, wherein the second connector comprises a plurality of male snap buttons and a plurality of female snap buttons.

4. The cover of claim 3, wherein the plurality of male snap buttons are disposed on an inner surface of the first end and the plurality of female snap buttons are disposed on an outer surface of the second end.

5. The cover of claim 1, wherein the sidewall is shaped to fit over a foot.

6. The cover of claim 1, wherein the sidewall is formed of a water impermeable material.

7. The cover of claim 1, wherein the elastic flexible band is made of a polymer.

8. The cover of claim 7, wherein the polymer is rubber.

9. A method of covering a cast of an injured animal comprising:
providing a cover comprising:
a tubular sidewall comprising a closed bottom end, an open top end formed by an upper rim, and a first inner edge and a second inner edge formed along a length of the tubular sidewall and terminating at the upper rim; and
an elastic flexible band attached to the upper rim and comprising a first end disposed near the first inner edge, and a second end disposed near the second inner edge;
placing the cover over the cast;
releasably connecting the first inner edge and the second inner edge together by a first connector, forming a water tight seal along the length of the tubular sidewall;
releasably connecting the first end and the second end of the elastic flexible band together by a second connector

10. The method of claim 9, wherein the first connector is a zipper running from the closed bottom end to the open top end and the second connector comprises a plurality of male snap buttons and a plurality of female snap buttons.
